(19) United States
(12) Patent Application Publication
(10) Pub. No.: US 2002/0095042 A1
(43) Pub. Date: Jul. 18, 2002

(54) Process for brominating the side chain of 4-methylbiphenyl derivatives substituted in the 2' position

(76) Inventors: Heinrich Schneider, Ingelheim (DE); Margarete Schneider, Ingelheim (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/945,600

(22) Filed: Sep. 4, 2001

(30) Foreign Application Priority Data
Apr. 12, 2000 (EP) PCT/EP00/03246

(51) Int. Cl. C07D 257/04; C07C 255/50; C07C 233/13

(52) U.S. Cl. 548/250; 558/411; 562/488; 564/171; 564/274

(57) ABSTRACT

A process for preparing 4-bromomethylbiphenyl derivatives substituted in the 2′ position of general formula (I)

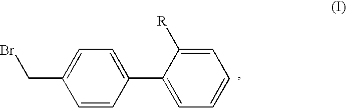

by brominating 4-methylbiphenyl derivatives substituted in the 2′ position of general formula (II)

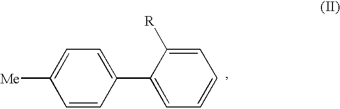

wherein:

R is CN, COOR^{1,} CONR¹R², or C(OR³)═NR⁴, or 5-tetrazolyl optionally substituted by benzyl, methyloxycarbonyl, ethyloxycarbonyl, 9-fluorenylmethyloxycarbonyl, 2,2,2-trichloroethyloxycarbonyl, benzyloxycarbonyl, tert-butyloxycarbonyl, allyloxycarbonyl, trichloroacetyl, trifluoroacetyl, or trityl;

R¹ and R² which are identical or different are hydrogen or C₁-C₆-alkyl;

R³ and R⁴ together are a C₂-C₃-alkylene bridge optionally mono-, di-, tri-, or tetra- substituted by one or more of the groups methyl, ethyl, n-propyl, isopropyl, n-butyl, isobutyl, tert-butyl, phenyl, or naphthyl,

wherein the brominating is carried out by means of an N-bromoimide or N-bromoamide in a solvent of organic carboxylic acid esters.

### RELATED APPLICATIONS

This application is a continuation of International Application No. PCT/EP00/03246, filed on Apr. 12, 2000, benefit of which is hereby claimed, pursuant to 35 U.S.C. §365(c) and §120.

### SUMMARY OF THE INVENTION

The invention relates to a process which can be used on an industrial scale for brominating the side chain of 4-methylbiphenyl derivatives substituted in the 2′ position with N-bromoimides or N-bromoamides to obtain 4-bromomethylbiphenyl derivatives of general formula (I) substituted in the 2′ position

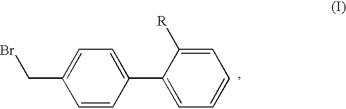

wherein R may have the meanings given in the specification and in the claims.

### BACKGROUND OF THE INVENTION

4-Bromomethylbiphenyl derivatives of general formula (I) substituted in the 2′ position are very valuable as intermediate products in the production of pharmaceutically useful active substances, particularly in the production of active substances for drugs which may be used as angiotensin-II-antagonists.

Processes for preparing biphenyl derivatives of general formula (I) are known from the prior art. EP-A-253310 proposes using N-bromosuccinimide (NBS hereinafter) in the presence of the radical forming agent azobis(isobutyronitrile) (AIBN hereinafter) in carbon tetrachloride at the boiling point of the solvent in order to react 4-methyl-2′-methoxycarbonylbiphenyl to obtain the corresponding bromomethyl derivative. For preparing 4-bromomethylbiphenyl derivatives, EP-A-553879 discloses bromination of the corresponding methylbiphenyls using NBS or N-bromophthalimide in the presence of a radical-forming agent in a halogenated solvent. EP-A-595150 discloses a method of preparing, for example, 4-bromomethyl-2′-cyanobiphenyl by bromination with NBS in the presence of benzoylperoxide in chlorobenzene as solvent.

The methods of brominating 4-methylbiphenyl derivatives substituted in the 2′ position known from the prior art do, however, have a number of drawbacks when attempts are made to carry out synthesis on an industrial scale. Thus, they generally use chlorinated hydrocarbons, which are known to be capable of causing liver and kidney damage, as solvents. Moreover, they are usually characterized by long reaction times when reactions are carried out on an industrial scale. However, lengthy reaction times lead to an increased formation of the bis-bromo products, which has a detrimental effect on the yield and purity of the desired mono-bromo derivatives.

The aim and objective of the present invention is therefore to provide an industrial-scale process for preparing 4-methylbiphenyl derivatives substituted in the 2′ position which overcomes the disadvantages encountered in the processes known from the prior art.

### DETAILED DESCRIPTION OF THE INVENTION

It has been found that, surprisingly, the abovementioned disadvantages of the bromination methods known from the prior art can be avoided if organic carboxylic acid esters are used as the solvents during bromination with N-bromoimides or N-bromoamides.

The present invention consequently relates to the preparation of 4-bromomethyl-biphenyl derivatives, substituted in the 2′ position, of general formula (I)

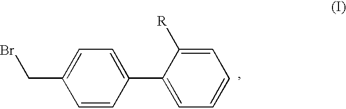

by reacting 4-methylbiphenyl derivatives, substituted in the 2′ position, of general formula (II)

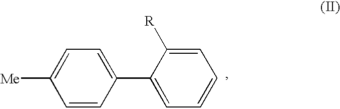

wherein:

R denotes CN, COOR¹, CONR¹R², or C(OR³)═NR⁴, or 5-tetrazolyl optionally substituted by benzyl, methyloxycarbonyl, ethyloxycarbonyl, 9-fluorenylmethyloxycarbonyl, 2,2,2-trichloroethyloxycarbonyl, benzyloxycarbonyl, tert-butyloxycarbonyl, allyloxycarbonyl, trichloroacetyl, trifluoroacetyl, or trityl;

R¹ and R² which may be identical or different denote hydrogen or C₁-C₆-alkyl, preferably C₁-C₄-alkyl; and

R³ and R⁴ together denote a C₂-C₃-alkylene bridge optionally mono-, di-, tri-, or tetra-substituted by one or more of the groups methyl, ethyl, n-propyl, isopropyl, n-butyl, isobutyl, tert-butyl, phenyl, or naphthyl,

characterized in that bromination is carried out by means of N-bromoimides or N-bromoamides in the presence or absence of a radical-forming agent in organic carboxylic acid esters as solvent.

A preferred process according to the invention is a process for preparing compounds of general formula (I) wherein:

R denotes CN, COOR¹, CONR¹R², or C(OR³)═NR⁴, or 5-tetrazolyl optionally substituted by benzyl, 9-fluorenylmethyloxycarbonyl, benzyloxycarbonyl, tert-butyloxycarbonyl, allyloxycarbonyl, or trityl;

R¹ and R² which may be identical or different denote hydrogen or C₁-C₄-alkyl; and

R³ and R⁴ together denote a C₂-C₃-alkylene bridge optionally mono- or disubstituted by methyl.

It is also preferred according to the invention to synthesize compounds of general formula (I) wherein R denotes CN, COOR¹, or 5-tetrazolyl, and R¹ denotes hydrogen or C₁-C₄-alkyl.

It is particularly preferred to synthesize compounds of general formula (I) wherein R denotes CN or COOR¹, and R¹ denotes hydrogen, methyl, ethyl, n-propyl, isopropyl, n-butyl, isobutyl, 2-butyl, or tert-butyl.

Of particular interest according to the invention is the synthesis of compounds of general formula (I) wherein R denotes CN, COOH, or COO-tert-butyl.

Suitable brominating agents include, in particular, the standard commercial N-bromoimides and N-bromoamides such as N-bromosuccinimide (NBS), N-bromophthalimide, N-bromoacetamide, N,N′-dibromo-5,5-dimethylhydantoin (DDH) or N,N′-dibromo-5,5-diphenylhydantoin. According to the invention it is preferable to use the brominating reagents NBS and DDH.

Suitable solvents include, in particular, organic carboxylic acid esters with 3 to 6 carbon atoms. It is preferable to use as the solvent a carboxylic acid ester selected from the group comprising methyl acetate, ethyl acetate, dimethyl carbonate or diethyl carbonate. It is particularly preferable to use methyl acetate and ethyl acetate as solvent. According to the invention, methyl acetate is of particular importance as the solvent.

The process according to the invention may be carried out in the presence or absence of a radical forming agent. If radical forming agents are used, according to the invention, standard commercial compounds of the azo-bis series may be used, e.g., AIBN or organic peroxides such as bis-benzoylperoxide. Preferably, AIBN is used as the radical forming agent.

The following method is used according to the invention to prepare 4-bromomethyl-biphenyl derivatives substituted in the 2′ position of general formula (I).

In a reaction apparatus of suitable dimensions, 100 mol % to 120 mol %, preferably 100 mol % to 110 mol %, most preferably about 105 mol % of brominating reagent and optionally 0 mol % to 2 mol % of radical forming agent, per 100 mol of methylbiphenyl derivative of general formula (II), are added to the solvent, the organic carboxylic acid ester, and the mixture is heated to boiling. In the course of the reaction (after about 70% to 80% of the reaction time) the brominating reagent goes into solution. As soon as the solution has become practically colorless, the solvent is mainly (about 50% to 90%) distilled off. The residue remaining is taken up in a mixture of an at least partially water-miscible solvent and the product is precipitated by the addition of water, while at the same time the imide or amide formed goes into solution. In the case of acid-sensitive products decomposition can be largely prevented by the addition of basic substances. Excesses of brominating reagent can be deactivated by the addition of reducing substances. After crystallization, the product is centrifuged, preferably washed with the same mixture of solvent and water, and dried.

The reaction times are generally in the range from 10 minutes to 8 hours. It has been found that the particular reaction time is strongly dependent on the purity of the compound of formula (II) used. Moreover, it has been found that the reaction times observed exhibit a strong dependency on concentration. To carry out the methods of synthesis according to the invention, the applicants normally used 30 to 100 liters of carboxylic acid ester, preferably 40 to 90 liters of carboxylic acid ester, most preferably about 50 to 80 liters of carboxylic acid ester per 100 mol of methylbiphenyl derivative of general formula (II). Depending on the amount of solvent used, which may also be outside the range specified above if it is desirable for the reaction to be faster or slower, it is possible to influence the reaction time decisively.

In addition to the advantages of the process according to the invention mentioned earlier, such as the absence of halogenated solvents, the short reaction times, and the reduced tendency to form the dibromo adducts, the process is characterized by a high space-time yield.

Moreover, the procedure according to the invention achieves a technically important improvement over other bromination processes with N-bromoimides or N-bromoamides known from the prior art. Since the solvent used according to the invention is partially water-miscible and the imides or amides formed in the course of bromination with N-bromoimides or N-bromoamides remain in solution in the mother liquor during the working up according to the invention in a mixture of water and another water-miscible solvent, laborious filtering of the mother liquor is no longer necessary.

The Examples which follow serve to illustrate methods of synthesis carried out by way of example for preparing 4-bromomethylbiphenyl derivatives substituted in the 2′ position. They should be regarded simply as possible procedures described by way of example, without restricting the invention to their content.

### EXAMPLE 1

241.2 kg of tert-butyl 4-methylbiphenyl-2′-carboxylate, 168.2 kg of NBS, 0.3 kg of AIBN, and 724 liters of methyl acetate are placed in a 1200-liter enamel stirring apparatus and the mixture is heated to 60° C. to 65° C. The reaction mixture begins to boil and after about 80% of the reaction time the NBS goes into solution. As soon as the solution has become practically colorless, 603 liters of solvent are distilled off under normal pressure, and then 482 liters of isopropanol, 121 liters of water, and 14.8 kg of sodium acetate (anhydrous) are added. The resulting mixture is cooled to 15° C. to 25° C. and stirred for a further 1 hour. The product precipitated is separated by centrifugation, washed with a mixture of 482 liters of isopropanol and 121 liters of water, and dried. Yield: 250 kg of tert-butyl 4-bromomethylbiphenyl-2′-carboxylate (80% of theory).

### EXAMPLE2

241.2 kg of tert-butyl 4-methylbiphenyl-2′-carboxylate, 168.2 kg of NBS, 0.3 kg of AIBN, and 724 liters of methyl acetate are placed in a 1200-liter enamel stirring apparatus and the mixture is heated to 60° C. to 65° C. The reaction mixture begins to boil and after about 80% of the reaction time the NBS goes into solution. As soon as the solution has become practically colorless, 603 liters of solvent are distilled off under normal pressure and then 482 liters of acetone, 121 liters of water, and 14.8 kg of sodium acetate (anhydrous) are added. The resulting mixture is cooled to 15° C. to 25° C. and stirred for a further 1 hour. The product precipitated is separated by centrifugation, washed with a mixture of 482 liters of acetone and 121 liters of water, and dried. Yield: 250 kg of tert-butyl 4-bromomethylbiphenyl-2′-carboxylate (80% of theory).

### EXAMPLE 3

173.7 kg of 4-methyl-2′-cyanobiphenyl, 168.2 kg of NBS, and 695 liters of methyl acetate are placed in a 1200-liter enamel stirring apparatus and the mixture is heated to 60° C. to 65° C. The reaction mixture begins to boil and after about 80% of the reaction time the NBS goes into solution. As soon as the solution has become practically colorless, 608 liters of solvent are distilled off under normal pressure and then 434 liters of methanol and 174 liters of water are added. The resulting mixture is cooled to 15° C. to 25° C. and stirred for a further 1 hour. The product precipitated is separated by centrifugation, washed with a mixture of 434 liters of methanol and 174 liters of water, and dried. Yield: 205 kg of 4-bromomethylbiphenyl-2′-cyanobiphenyl (83.7% of theory).

### EXAMPLE 4

268.0 kg of tert-butyl 4-methylbiphenyl-2′-carboxylate, 157.3 kg of DDH, 1.6 kg of AIBN, and 572 liters of methyl acetate are placed in a 1200-liter enamel stirring apparatus and the mixture is heated to 60° C. to 65° C. As soon as the solution has become practically colorless, 458 liters of solvent are distilled off under normal pressure and then 530 liters of isopropanol, 148 liters of water, and 16.4 kg of sodium acetate (anhydrous) are added. The resulting mixture is cooled to 15° C. to 25° C. and stirred for a further 1 hour. The product precipitated is separated by centrifugation, washed with a mixture of 530 liters of acetone and 148 liters of water, and dried. Yield: 295 kg of tert-butyl 4-bromomethylbiphenyl-2′-carboxylate (85% of theory).

### EXAMPLE 5

212 kg of 4-methylbiphenyl-2′-carboxylic acid, 195.8 kg of NBS, 1.6 kg of AIBN, and 530 liters of methyl acetate are placed in a 1200 liters enamel stirring apparatus and the mixture is heated to 60° C. to 65° C. The reaction mixture begins to boil and after about 80% of the reaction time the NBS goes into solution. As soon as the solution has become practically colorless, 424 liters of solvent are distilled off under normal pressure and then 424 liters of methanol and 106 liters of water are added. The resulting mixture is cooled to 15° C. to 25° C. and stirred for a further 1 hour. The product precipitated is separated by centrifuigation, washed with a mixture of 424 liters of methanol and 106 liters of water, and dried. Yield: 240 kg of 4-bromomethylbiphenyl-2′-carboxylic acid (82.5% of theory).

We claim:

1. A process for preparing 4-bromomethylbiphenyl derivatives substituted in the 2′ position of general formula (I) 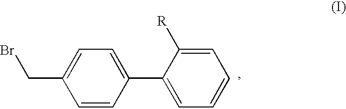
by brominating 4-methylbiphenyl derivatives substituted in the 2′ position of general formula (II) 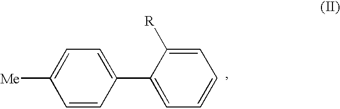
wherein:
R is CN, COOR¹, CONR¹R², or C(OR³)═NR⁴, or 5-tetrazolyl optionally substituted by benzyl, methyloxycarbonyl, ethyloxycarbonyl, 9-fluorenylmethyloxycarbonyl, 2,2,2-trichloroethyloxycarbonyl, benzyloxycarbonyl, tert-butyloxycarbonyl, allyloxycarbonyl, trichloroacetyl, trifluoroacetyl, or trityl;
R¹ and R² which are identical or different are hydrogen or C₁-C₆-alkyl;
R³ and R⁴ together are a C₂-C₃-alkylene bridge optionally mono-, di-, tri-, or tetra- substituted by one or more of the groups methyl, ethyl, n-propyl, isopropyl, n-butyl, isobutyl, tert-butyl, phenyl, or naphthyl,
wherein the brominating is carried out by means of an N-bromoimide or N-bromoamide in a solvent of organic carboxylic acid esters.

2. The process according to claim 1, wherein R¹ and R² which are identical or different are each hydrogen or C₁-C₄-alkyl.

3. The process according to claim 1, wherein:
R is CN, COOR¹, CONR¹R², or C(OR³)═NR⁴, or 5-tetrazolyl optionally substituted by benzyl, 9-fluorenylmethyloxycarbonyl, benzyloxycarbonyl, tert-butyloxycarbonyl, allyloxycarbonyl, or trityl;
R¹ and R² which are identical or different are each hydrogen or C₁-C₄-alkyl;
R³ and R⁴ together are a C₂-C₃-alkylene bridge optionally mono- or disubstituted by methyl.

4. The process according to claim 1, wherein:
R is CN, COOR¹, or 5-tetrazolyl; and
R¹ is hydrogen or C₁-C₄-alkyl.

5. The process according to claim 1, wherein:
R is CN or COOR¹; and
R¹ is hydrogen, methyl, ethyl, n-propyl, isopropyl, n-butyl, isobutyl, 2-butyl, or tert-butyl.

6. The process according to claim 1, wherein R is CN, COOH, or COO-tert-butyl.

7. The process according to one of claims 1 to 6, wherein the N-bromoimide or N-bromoamide is selected from the group consisting of: N-bromosuccinimide (NBS), N-bromophthalimide, N-bromo-N-methylacetamide, N,N′-dibromo-5,5-dimethylhydantoin (DDH), and N,N′-dibromo-5,5-diphenylhydantoin.

8. The process according claim 7, wherein the N-bromoimide or N-bromoamide is selected from the group consisting of: N-bromosuccinimide (NBS) and N,N′-dibromo-5,5-dimethylhydantoin (DDH).

9. The process according to one of claims 1 to 6, wherein the organic carboxylic acid ester solvent is a carboxylic acid ester having 3 to 6 carbon atoms.

10. The process according to claim 7, wherein the organic carboxylic acid ester solvent is a carboxylic acid ester having 3 to 6 carbon atoms.

11. The process according to claim 8, wherein the organic carboxylic acid ester solvent is a carboxylic acid ester having 3 to 6 carbon atoms.

12. The process according to claim 9, wherein the organic carboxylic acid ester solvent is selected from the group consisting of: methyl acetate, ethyl acetate, dimethyl carbonate, and diethyl carbonate.

13. The process according to claim 10, wherein the organic carboxylic acid ester solvent is selected from the group consisting of: methyl acetate, ethyl acetate, dimethyl carbonate, and diethyl carbonate.

14. The process according to claim 11, wherein the organic carboxylic acid ester solvent is selected from the group consisting of: methyl acetate, ethyl acetate, dimethyl carbonate, and diethyl carbonate.

15. The process according to claim 12, wherein the organic carboxylic acid ester solvent is selected from the group consisting of: methyl acetate and ethyl acetate.

16. The process according to claim 13, wherein the organic carboxylic acid ester solvent is selected from the group consisting of: methyl acetate and ethyl acetate.

17. The process according to claim 14, wherein the organic carboxylic acid ester solvent is selected from the group consisting of: methyl acetate and ethyl acetate.

18. The process according to claim 1, wherein, during working up, the imide or amide formed is dissolved in a mixture of water and a water-miscible solvent in the mother liquor.
